(19) United States
(12) Patent Application Publication
(10) Pub. No.: US 2023/0047221 A1
(43) Pub. Date: Feb. 16, 2023

(54) STERILITY HEADSET DEVICE FOR SURGICAL AND PARA-SURGICAL PROCEDURES

(71) Applicants: Enrico PEIRETTI, Cagliari CA (IT); Ranieri PEIRETTI, Cagliari CA (IT); Maria Giuseppina CARBONI, Cagliari CA (IT)

(72) Inventors: Enrico PEIRETTI, Cagliari CA (IT); Ranieri PEIRETTI, Cagliari CA (IT); Maria Giuseppina CARBONI, Cagliari CA (IT)

(21) Appl. No.: 17/797,168

(22) PCT Filed: Feb. 13, 2020
(86) PCT No.: PCT/IB2020/051183

§ 371 Date: Aug. 3, 2022

(51) Int. Cl. A61B 90/40; A61F 9/007

(52) U.S. Cl. CPC A61B 90/40; A61F 9/007; A61B 2090/401

(57) ABSTRACT

The present invention relates to a device adapted to create a sterile environment located at the patient’s head and face level, necessary during surgical and para-surgical procedures, for example in the dermatological, aesthetic-plastic, reconstructive and ophthalmic fields. Said device, small in size and easily transportable, allows to create a sterile environment located in the surgical area without the need for an operating theatre.

### STATE OF THE ART

There are numerous traumatic or pathological states that involve the area of the face and head, such as burns, which is important that remain under aseptic conditions as bacterial contamination can cause important damage to the individual, sometimes irreparable. Additionally, many surgical procedures carried out at the face and head levels, sometimes treated on an outpatient basis, would benefit from being addressed in a sterile environment or nearing sterility, leading to a lower risk for the operator and better guarantees for the patient. If we consider the ophthalmic field, there are many eye diseases that need a sterile and controlled environment in order to complete a series of more or less invasive therapies, such as, for example, intraocular injection therapies, which are becoming more and more common and frequent. These injections to be done inside the eye are carried out, understandably, through the use of sterile material, as well as according to the guidelines drawn up by the Societa Oculistica Italiana (Italian Ophthalmology Society), in an operating theatre, therefore in an environment as sterile as possible, precisely to ensure greater safety of the procedure.

With the idea of being able to overcome the need to have an entire, completely sterile, operating theatre to carry out micro-surgery procedures and small surgeries, alternative solutions have been proposed, such as, for example, some parallelepiped structures of various sizes (e.g. 2 - 4 m long and about 2.5 m high), which can accommodate the operator and at least half of the patient’s body. Inside these structures, the air is filtered and diffused with a vertical laminar flow over the operating surface and the serving tables, in order to make sterile the portion of the room in which the surgery is carried out.

However, these structures are expensive, cumbersome and must be permanently installed in a precise position; moreover, they need about five minutes of operation of the laminar airflow to create the correct sterile environment therein.

Another possible approach to this technical problem is a portable trolley for surgical instruments, equipped with a high efficiency filter (HEPA - High Efficiency Particulate Air). A laminar airflow passes through this filter and determines the formation of a sterile area directly on the table used for surgical instruments and on the surgical area. However, the sterile area created by this device is not exactly circumscribed, no visible barrier delimits its boundaries and the surgical area may not be completely and adequately protected from contamination. Furthermore, as structures are completely open, any fall of non-sterile material within the surgical area could adversely affect their ability to act.

The need to have a more economical, manageable and easily transportable system available that is both safe and efficient in order to create a sterile environment in a circumscribed area of space in which to carry out surgical and para-surgical procedures, is still felt. A system specifically aimed at procedures to be carried out in the ophthalmic field and/or the field related to microsurgery at the face level, could be of particular interest.

### OBJECTS OF THE INVENTION

In the light of the above, object of the present invention is to provide a portable device capable of creating a sterile environment in a precise and circumscribed area of space in which surgical and para-surgical procedures are carried out, in particular at the eye and head levels, the face in particular, of the patient undergoing surgery.

Further object of the invention is to allow surgical and para-surgical procedures, in particular ophthalmic and/or of the head, to be carried out in a microbiologically aseptic environment even in the outpatient clinic, without the need for an operating theatre. Additionally, object of the present invention is using said device during surgical and para-surgical procedures involving the face and head, with particular attention to the noble structures of the face, such as the eyes.

Further object of the present invention is the ability to use said device in the field of prevention and safety in the workplace, sport, school environments and more, because it allows to immediately secure, within an aseptic environment, the head of a subject who has suffered a trauma, and be able to transport him while maintaining the sterility of the wound.

### DESCRIPTION OF THE FIGURES

FIG. 1 shows a schematic representation of a non-limiting example of the device object of the invention (A) in closed (a) and open (b) configurations. The following are shown in detail: a front closable portion (1), two side openings (2) on said front closable portion and two systems of fans coupled to an air sterilisation system, which require a laminar flow (3).

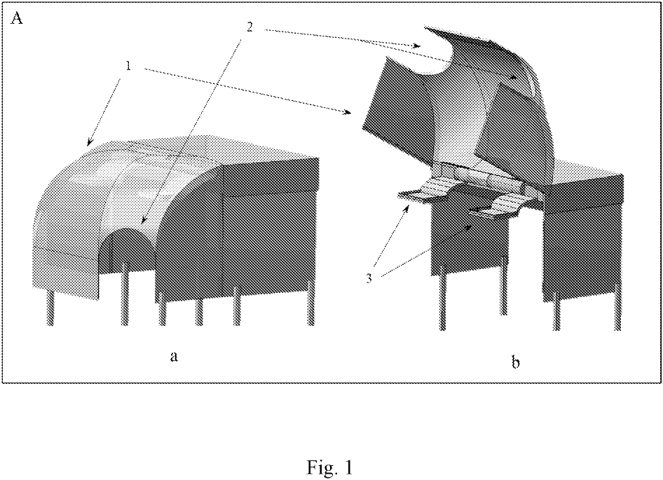

FIG. 2 shows a different possible configuration (B) of the device (A) object of the invention, which can be mounted on a seat. The following are also present in this configuration: a front closable portion (1), two side openings (2) on said front closable portion and two systems of fans coupled to an air sterilisation system, which require a laminar flow (3).

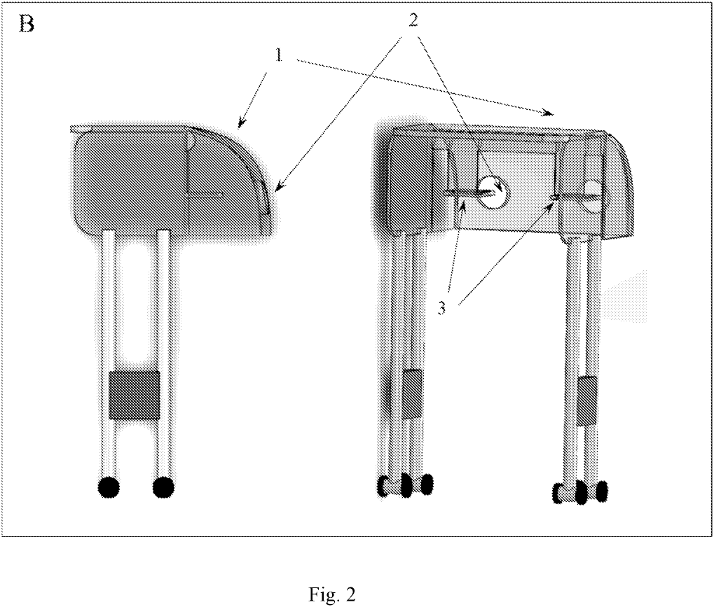

### DESCRIPTION OF THE INVENTION

The subject-matter of the present invention is a device (A) adapted to create a sterile area that can be located at a subject’s head level, characterised by the presence of at least one front closable portion (1), at least one system for sterilising the incoming air and at least one pumping system for forming a laminar airflow within said device, which is useful during surgical and para-surgical procedures.

Said device (A) is small in size and easily transportable, and allows to create a sterile environment located at a patient’s head level, in which surgical and para-surgical surgeries can be carried out without necessarily needing to have an operating theatre available. In this document, “sterile environment” means, for example, an environment that meets the present national/international classifications/regulations currently recognised. In fact, the main means of transport of micro-organisms contained in the air is precisely the same particulate present in the environment. The higher the particulate the higher the chances of having a high bacterial load of the air. The last standard published on the filing date of the present invention by UNI (11425:2011) defines that it is appropriate that the Operating Theatre is, under At-Rest conditions (stopped surgical activity), at least in class:
ISO5 for complex surgeries, such as transplants, implants, neurosurgery, oncology, orthopaedics, lasting more than 60 minutes;
ISO7 for surgeries without implantation of foreign materials, such as minimally invasive, vascular, obstetric, ophthalmic surgeries;
ISO8 for short-term surgeries, such as visceral surgery, day surgery, urology.

Said device comprises several parts combined with each other to give a system that has a shape similar to a headset device, is mounted on a movable structure, possibly on wheels, and can be coupled to a headrest cushion. Said headrest cushion, of washable and sterilisable material according to the applicable standards, is useful to support the patient’s head in the proper way in order to both carry out the surgical or para-surgical procedure, and to ensure that the position of the patient’s head is maintained within the sterile area. It also ensures the subject’s comfort during the procedure.

In a different configuration, which can be used in combination with a seat, e.g. an outpatient clinic chair or an office chair, said device also has a shoulder rest.

In particular, the device of the invention is characterised by the presence of at least one front closable portion, at least one system for sterilising the incoming air and at least one pumping system for forming a laminar airflow within said device.

The whole device, including the movable front closable portion (1), is made of a transparent, washable and sterilisable material according to the applicable standards, e.g. Plexiglas. Said closable portion is rotated over the patient’s head and closed at the subject’s neck level. Possibly, a small, also sterile, cloth can be applied on the edge of said closable portion so as to create a protection and greater comfort for the patient when the structure is closed.

The two openings (2), for the entrance of the operator’s hands, are positioned on the top and rear sides of the headset device in the supine version and at the side of the patient’s face in the configuration that can be coupled to the seat. Said openings allow the operator to introduce the hands and instruments into the procedure area and then carry out the surgery.

The device of the invention also comprises at least one system that allows the filtration of incoming air and is capable of retaining micro-organisms. A non-limiting example of said filter is represented by a HEPA (High Efficiency Particulate Air Filter) filter, with overall efficiency of 99.995% (class 14, according to UNI EN 1822 standards) normally used in laminar flow hoods to keep the environment clean, sometimes coupled to a pre-filter. A second possible type of filter is the ULPA (Ultra Low Penetration Air) filter. Preferably, a HEPA filter is used.

The device of the invention comprises, coupled to said at least one system for filtering the incoming air and at least one system for pumping the outgoing filtered air, according to a vertical laminar flow directed to the operating field (3). In a preferred configuration, the device object of the invention can be equipped with a dual ventilation system for air recycling and ejection, respectively. For example, a battery-powered fan with voltage between 12 and 24 Volts (V), power in the order of 2 Watts (W), can be used as a reference system. In particular, a fan with the dimensions 60 x60 x25 mm with voltage equal to 12 V, a current value of 200 mA, power of 2.40 W and weight of 57 g, can be used.

Said air pumping and intake system being fed, preferably, by means of at least one, preferably two batteries, one per part, rechargeable or not, or thanks to an electrical connection.

According to a preferred aspect, the device, which is the subject-matter of the invention, will begin to filter the incoming air and to produce laminar flow of sterile air on the operating field by means of a switch once the movable portion is placed in the closed position (a) and can be turned off through the same switch once opened (b). If necessary or preferable, the laminar flow can also be kept running in position (b).

Thanks to the limited and well-contained volume of air present inside the device in question, the creation of the sterile environment therein, determined by the laminar flow of filtered air, will take place in a few tens of seconds.

Also, according to the present invention, said device (A), including its particular configuration (B), is advantageously used for carrying out surgical and para-surgical procedures, in particular of the head. Advantageously, said surgical and para-surgical procedures are procedures in the dermatology, plastic surgery and ophthalmic surgery fields.

A method for carrying out surgical and para-surgical procedures, in particular of the head and face, as well as of the noble structures of the face, such as the eyes, is therefore object of the present invention.

A further subject-matter of the present invention is the use of the device of the invention in the field of prevention and safety in the workplace, school, sport environments and more, because it can allow to immediately secure, within an aseptic environment, the head of a subject who has suffered a trauma, and be able to transport him while maintaining the sterility of the wound.

1. Device adapted to create a sterile area that can be located at a subject’s head level, characterised by the presence of at least one front closable portion (1), at least one system for sterilising the incoming air and at least one pumping system for forming a laminar airflow within said device.

2. Device according to claim 1, characterised in that said device can be coupled to a headrest cushion or a seat and a neck support.

3. Device according to claims 1 or 2, characterised in that said device is made of sterilisable material.

4. Device according to any one of the preceding claims, characterised in that at least one front closable portion (1) is transparent and provides at least one opening (2) which allows the insertion of the operator’s hands and the instruments into the sterile operating field.

5. Device according to any one of the preceding claims, characterised in that said at least one front closable portion (1) is tied to the top part of said device by means of hinges that allow its rotation to switch from closed to open configuration and vice versa.

6. Device according to any one of the preceding claims, characterised in that said at least one system for sterilising the incoming air consists of an air filter capable of retaining micro-organisms.

7. Device according to any one of the preceding claims, characterised in that said at least one pumping system for forming a laminar airflow (3) is connected to said at least one system for sterilising the incoming air.

8. Device according to any one of the preceding claims, characterised in that said at least one pumping system for forming a laminar airflow (3) is a fan.

9. Device according to any one of the preceding claims, characterised in that said at least one pumping system for forming a laminar airflow is positioned on the top sides of said device.

10. Device according to any one of the preceding claims, characterised in that said at least one pumping system for forming a laminar airflow (3) is activated by means of a special switch or automatically when said at least one front closable portion (1) is in closed position.

11. Device according to any one of the preceding claims, characterised in that at least one pumping system for forming a laminar airflow (3) is stopped by means of a special switch or automatically when said at least one front closable portion (1) is in open position.

12. Device according to any one of the preceding claims, characterised in that the environment therein is sterile already a few tens of seconds after its activation.

13. Use of the device according to claim 1 for carrying out surgical and para-surgical procedures, in particular of the head.

14. Use of the device according to claim 13, characterised in that said surgical and para-surgical procedures are procedures in the ophthalmic field.

15. Use of the device according to claim 1 for maintaining face or head wounds under sterile conditions in emergency situations.
